(19) United States
(12) Patent Application Publication
(10) Pub. No.: US 2025/0011409 A1
(43) Pub. Date: Jan. 9, 2025

(54) VEGF-BINDING PROTEIN AND USE THEREOF

(71) Applicant: LINNO PHARMACEUTICALS INC., Shanghai (CN)

(72) Inventors: Zhaozhong HAN, Shanghai (CN); Hongya PAN, Shanghai (CN)

(73) Assignee: LINNO PHARMACEUTICALS INC., Shanghai (CN)

(21) Appl. No.: 18/711,199

(22) PCT Filed: Nov. 24, 2021
(86) PCT No.: PCT/CN2021/132760

§ 371 Date: May 17, 2024

(51) Int. Cl. C07K 16/22

(52) U.S. Cl. CPC C07K 16/22; C07K 2317/22; C07K 2317/24; C07K 2317/569; C07K 2317/73; C07K 2317/76

(57) ABSTRACT

The present disclosure provides an isolated antigen-binding protein, having one or more properties selected from the group consisting of: 1) an ability of specifically binding to VEGFA (Vascular Endothelial Growth Factor A); 2) an ability of preventing VEGF from binding its corresponding receptor (s); and 3) an ability of inhibiting VEGF-driven biological functions. The present application also provides bi-paratopic antigen-binding proteins and polypeptides comprising the isolated antigen-binding protein and their use thereof.

### BACKGROUND OF THE INVENTION

Vascular Endothelial Growth Factor (VEGF) is a growth factor capable of promoting the division and proliferation of endothelial cells, promoting the formation of new blood vessels, and improving vascular permeability. It exercises such biological activities through binding to appropriate growth factor receptor(s) and activating intracellular signaling pathway(s). Within tumor mass, multiple types of cells including tumor cells, and tumor-infiltrating macrophages and mast cells can secrete high levels of VEGF that stimulate sprouting of surrounding blood vessels towards tumor mass, homing, proliferation and migration of endothelial progenitor cells, and collectively induce angiogenesis to support continuous growth of tumor mass. High levels of VEGF also improve vascular permeability, promote fibroblast invasion, fibrous deposition and tumor stroma formation that contributes to drug resistance and tumor metastasis. Thus, VEGF inhibition has been studied as one of the most promising options for tumor treatment. The VEGF family includes VEGF-A, VEGF-B, VEGF-C, VEGF-D and PIGF. VEGF-A is a homodimeric glycoprotein with a molecular weight of 45 KD. As the most specific and most critical angiogenesis factor among the family members, VEGF-A primarily binds VEGFR-2 to activate the downstream signaling pathways for exercising its biological activities.

VEGF-A inhibiting antibodies and tyrosine kinase inhibiting compounds have been exploited as therapeutics for VEGF-driven diseases like tumor or wet age-related macular degeneration (wAMD). There is a need to obtain the VEGFA-binding proteins with various bioactivities for treating VEGFA-associated diseases.

### SUMMARY OF THE INVENTION

The present disclosure provides an antigen-binding protein capable of binding VEGF-A, and use thereof.

In one aspect, the present application provides an isolated antigen-binding protein, having one or more properties selected from the group consisting of: 1) an ability of specifically binding to VEGF-A (Vascular Endothelial Growth Factor A); 2) an ability of preventing VEGF-A from binding to its corresponding receptor(s); and 3) an ability of inhibiting VEGF-driven biological functions.

In some embodiments, the VEGF-driven biological function comprises angiogenesis.

In some embodiments, the VEGF-A comprises human VEGF-A.

In some embodiments, the isolated antigen-binding protein comprises antibody or its antigen-binding fragment.

In some embodiments, the antibody comprises single domain antibody, monoclonal antibody, single strand antibody, chimeric antibody, polyspecific antibody, humanized antibody and fully human antibody.

In some embodiments, the antigen-binding fragment comprises Fab, Fab′, F(ab)2, F(ab′)2, sdAb, Fv and ScFv fragment and bi-paratopic antigen-binding protein.

In some embodiments, the isolated antigen-binding protein is a single domain antibody (sdAb) or its antibody fragment.

In some embodiments, the isolated antigen-binding protein comprises a HCDR3, the HCDR3 comprises an amino acid sequence as set forth in any one of SEQ ID NO: 1, SEQ ID NO: 4, SEQ ID NO: 7, and SEQ ID NO: 10.

In some embodiments, the isolated antigen-binding protein comprises a HCDR2, the HCDR2 comprises an amino acid sequence as set forth in any one of SEQ ID NO: 2, SEQ ID NO: 5, SEQ ID NO: 8, and SEQ ID NO: 11.

In some embodiments, the isolated antigen-binding protein comprises a HCDR1, the HCDR1 comprises an amino acid sequence as set forth in any one of SEQ ID NO: 3, SEQ ID NO: 6, SEQ ID NO: 9, and SEQ ID NO: 12.

In some embodiments, the isolated antigen-binding protein comprises a HCDR1, a HCDR2, and a HCDR3, wherein the HCDR1, the HCDR2 and the HCDR3 comprises the amino acid sequences selected any one from the group consisting of: 1) the HCDR1 comprises an amino acid sequence as set forth in SEQ ID NO: 3, the HCDR2 comprises an amino acid sequence as set forth in SEQ ID NO: 2, and the HCDR3 comprises an amino acid sequence as set forth in SEQ ID NO: 1; 2) the HCDR1 comprises an amino acid sequence as set forth in SEQ ID NO: 6, the HCDR2 comprises an amino acid sequence as set forth in SEQ ID NO: 5, and the HCDR3 comprises an amino acid sequence as set forth in SEQ ID NO: 4; 3) the HCDR1 comprises an amino acid sequence as set forth in SEQ ID NO: 9, the HCDR2 comprises an amino acid sequence as set forth in SEQ ID NO: 8, and the HCDR3 comprises an amino acid sequence as set forth in SEQ ID NO: 7; and 4) the HCDR1 comprises an amino acid sequence as set forth in SEQ ID NO: 12, the HCDR2 comprises an amino acid sequence as set forth in SEQ ID NO: 11, and the HCDR3 comprises an amino acid sequence as set forth in SEQ ID NO: 10.

In some embodiments, the isolated antigen-binding protein comprises an amino acid sequence as set forth in any one of SEQ ID NO: 13-16.

In another aspect, the present application provides a bi-paratopic antigen-binding protein, wherein the bi-paratopic antigen-binding protein comprises a first antigen-binding domain, and a second antigen-binding domain, wherein the first antigen-binding domain and/or the second antigen binding domain comprises said antigen-binding protein.

In some embodiments, the first antigen-binding domain and said second antigen-binding domain of the bi-paratopic antigen-binding protein target the same antigen.

In some embodiments, the bi-paratopic antigen-binding protein comprises a HCDR3, said HCDR3 comprises an amino acid sequence as set forth in any one of SEQ ID NO: 1, SEQ ID NO: 4, SEQ ID NO: 7, and SEQ ID NO: 10.

In some embodiments, the bi-paratopic antigen-binding protein comprises an amino acid sequence as set forth in any one of SEQ ID NO: 17-19.

In another aspect, the present application provides a polypeptide, the polypeptide comprises any one of the isolated antigen-binding protein of the present application or any one of the bi-paratopic antigen-binding protein of the present application.

In some embodiments, the polypeptide further comprises one or more other functional domain(s).

In some embodiments, the functional domain of the polypeptide comprises a Fc region.

In some embodiments, the Fc region comprises a human Fc region.

In some embodiments, the functional domain of the polypeptide comprises a complement-inhibiting entity.

In some embodiments, the functional domain of the polypeptide comprises a human complement H (CFH) fragment.

In some embodiments, the polypeptide comprises an amino acid sequence as set forth in any one of SEQ ID NO: 21, and SEQ ID NO: 23.

In another aspect, the present application provides one or more isolated nucleic acid molecules, encoding any one of the isolated antigen-binding proteins of the present application, any one of the bi-paratopic antigen-binding protein of the present application, or any one of the polypeptides of the present application.

In another aspect, the present application provides a vector, the vector comprises the nucleic acid molecules of the present application.

In another aspect, the present application provides a cell, the cell comprises he nucleic acid molecules or the vector of the present application.

In another aspect, the present application provides a pharmaceutical composition, the pharmaceutical composition comprises any one of the isolated antigen-binding proteins, any one of the bi-paratopic antigen-binding proteins of the present application, any one of the polypeptides of the present application.

In another aspect, the present application provides a use of the isolated binding-protein of the present application, the bi-paratopic antigen-binding protein of the present application, the polypeptide of the present application, and/or the pharmaceutical composition of the present application, in preparation of a drug, and said drug is used for preventing and/or treating a disease.

In some embodiments, the disease comprises a tumor, an age-related macular degeneration, or a VEGFA-driven pathogenic process.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates the purity of VHH-Fcs analysis on non-reducing SDS-PAGE.

FIG. 2 illustrates VHH-Fc binding to hVEGFA121 and Receptor Blocking Activity.

FIG. 3 illustrates the epitope binning results.

FIG. 4 illustrates the purity of Bi/Tri-specific VHH-Fc analysis on SDS-PAGE.

FIG. 5 illustrates the Bi/Tri-specific VHH-Fc Binding to hVEGFA121 and Receptor Blocking Activity.

FIG. 6 illustrates the humanized VHH-Fc binding to hVEGFA121 and Receptor Blocking Activity.

FIG. 7 illustrates the humanized Bi-specific VHH-Fc binding to hVEGFA121 or mVEGF120 and Receptor Blocking Activity.

FIG. 8 illustrates RBA assays of hVEGFA (165 and 121), mVEGFA (120) on human VEGFR2.

FIG. 9 illustrates the inhibition of HUVEC cell proliferation.

FIG. 10 illustrates the dual functional recombinant proteins inhibiting VEGF-driven angiogenesis and factor H-regulated complement activation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In the present application, the term “VEGF”, or vascular endothelial growth factor, generally refers to a family of signaling proteins that can stimulate for example angiogenesis, vasculogenesis and/or lymphangiogenesis. Members of the VEGF family include VEGF-A, VEGF-B, VEGF-C, VEGF-D, and PIGF (Placental Growth Factor). In the present application, the term can include all forms of VEGF molecules, for example, their variants, or fragments.

In the present application, the term “antigen-binding protein” generally refers to a protein that is able to bind one or more antigen. In the present application, the term can include an antibody or antibody fragments. In some embodiments, the term can include a single domain antibody or a VHH fragment.

In the present application, the term “antibody” refers generally to a polypeptide molecule capable of specifically recognizing and/or neutralizing a specific antigen. For example, the antibody can include a heavy (H) chain and/or a light (L) chain (e.g., it can be an immunoglobulin that can include two heavy chains and/or light chains), and include any molecule including its antigen-binding fragment. The term “antibody” can include monoclonal antibody, antibody fragments or antibody derivative, including but not limited to single domain antibody, human antibody (fully human antibody), humanized antibody, chimeric antibody, single strand antibody (e.g., scFv), and antigen-binding fragment (e.g., Fab, Fab′ and (Fab)2 fragments). Each heavy chain can be composed of heavy chain variable regions (VHs) and heavy chain constant regions. Each light chain can be composed of light chain variable regions (VLs) and light chain constant regions. VH and VL regions can be further divided into hypervariable regions called complementary determining regions (CDRs), which are dispersed in more conserved regions called framework regions (FRs). Each of VH and VL can be composed of three CDRs and four FR regions, which can be arranged from the amino terminus to the carboxyl terminus in the order of FR1, CDR1, FR2, CDR2, FR3, CDR3 and FR4. The variable regions of heavy chain and light chain include binding domains that interact with the antigen. The constant regions of the antibody can mediate the binding of the immunoglobulin to the host tissues or factors.

In the present application, the term “antigen-binding fragment” refers generally to one or more fragments in the antibody that function to specifically bind to the antigen. The antigen-binding function of the antibody can be achieved by a full-length fragment of the antibody. And the antigen-binding function of the antibody can also be achieved by the following: a heavy chain including Fv, ScFv, dsFv, Fab, Fab′ or F(ab′)2 fragments, or a light chain including Fv, ScFv, dsFv, Fab, Fab′ or F(ab′)2 fragments. (1) Fab fragment, that is a monovalent fragment composed of VL, VH, CL and CH domains; (2) F(ab′)2 fragment, that is a divalent fragment including two Fab fragments linked via a disulfide bond in the hinge region; (3) Fd fragment composed of VH and CH domains; (4) Fv fragment composed of VL and VH domains of a single arm of the antibody; (5) dAb fragment composed of VH domains (Ward et al., (1989) Nature 341: 544-546); (6) isolated complementary determining regions (CDRs); and (7) a combination of two or more CDRs that are optionally linked via a linker. Moreover, it can also include a monovalent single-strand molecule Fv (scFV) formed by pairing of VL and VH (see, Bird et al., (1988) Science 242: 423-426; and Huston et al., (1988) Proc. Natl. Acad. Sci. 85: 5879-5883).

In the present application, the term “VHH” also known as VHH domains, VHH antibody fragments, and VHH antibodies, have originally been described as the antigen binding immunoglobulin (variable) domain of “heavy chain antibodies”. For example, having the structure of FR1-CDR1-FR2-CDR2-FR3-CDR3-FR4 and specifically binding to an epitope without requiring the presence of a second immunoglobulin variable domain.

In the present application, the term “monoclonal antibody” refers generally to a population of substantially homogeneous antibodies, that is, various antibodies contained in the population are the same except potential naturally occurring mutations present in a trace amount. The monoclonal antibody can be highly specific, and directly target a single antigenic site. The monoclonal antibody can be prepared by hybridoma technology or produced in bacteria, eukaryotic animals or plant cells by using recombinant DNA methods. The monoclonal antibody can also be obtained from a phage antibody library, by using a technology as described in, e.g., Clackson et al., Nature, 352:624-628 (1991) and Marks et al., Mol. Biol., 222:581-597 (1991).

In the present application, the term “single strand antibody” (scFv) refers generally to a molecule including antibody heavy chain variable regions and light chain variable regions. For example, the scFv can be formed by linking an antibody heavy chain variable region to a light chain variable region via a joint molecule (linker) (e.g., a connecting peptide).

In the present application, the term “chimeric antibody” refers generally to an antibody in which a part of the amino acid sequences of the heavy chain or the light chain is homogeneous to the corresponding amino acid sequence in an antibody derived from specific species or belongs to a certain class, while the other part of the chain is homogeneous to the corresponding sequence in another species. For example, the variable regions of the light chain and the heavy chain can be derived from the variable region of the antibody of an animal species (e.g., mice, rats, and the like), while the constant part is homogeneous to the sequence of an antibody derived from another species (e.g., human). For example, to obtain a chimeric antibody, the variable region can be produced by using non-human B cell or hybridoma cells, while the constant region combined therewith is derived from human. Since the constant region of the chimeric antibody can be derived from human, the chimeric antibody is less likely to elicit an immune response when injected than the antibody that uses a constant region of non-human origin.

In the present application, the term “humanized antibody” refers generally to an antibody that includes less sequence derived from non-human immunoglobulin, so as to reduce the immunogenicity when a heterogeneous antibody is introduced into a human. For example, it is feasible to use CDR transplant (Jones et al., Nature 321:522 (1986)) and its variant; including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154) and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312), surface rendering (U.S. Pat. No. 5,639,641) and other technical means to humanize the binding domain of non-human source. If other regions, e.g., the hinge region and the constant region domain, are also derived from a non-human origin, these regions can also be humanized.

In the present application, the term “fully human antibody” refers generally to a full human antibody, namely, both the constant region and the variable regions of the antibody are derived from human. The fully human antibody can be achieved by phage antibody library technology, production of a humanized antibody by transgenic mice, ribosome display technology, EBV transformed B cell cloning technology, single B cell cloning and other technologies, and the like.

The term “bi-paratopic antigen-binding protein” generally refers to an antigen-binding molecule comprising a first antigen-binding domain and a second antigen-binding domain, wherein the two antigen-binding domain binds to two different epitopes, for example, non-overlapping epitopes of the respective antigen. In some embodiments, the first antigen-binding domain and the second antigen-binding domain may target the same antigen. For example, the first antigen-binding domain and the second antigen-binding domain target different epitopes of the same antigen. The part of an antigen-binding protein that recognize the epitope is called a paratope.

In the present application, the term “tumor” refers generally to a physiological condition characterized by dysregulation of cell proliferation or survival. The tumor can include all the known cancers and tumor conditions, no matter their characteristics are malignant, benign, soft tissue, or solids, and can include cancers of all stages and grades including pre-metastatic and post-metastatic cancers. The tumor can further include one or more tumor cells

In the present application, the term “nucleic acid molecule” refers generally to isolated forms of nucleotides, deoxyribonucleotides, or ribonucleotides of any length that are isolated from their natural environment or artificially synthesized or their analogs.

In the present application, the term “vector” generally refers to a nucleic acid molecule capable of self-replication in a suitable host, which transfers the inserted nucleic acid molecule into the host cell and/or between the host cells. The vector can include a vector mainly used for inserting DNA or RNA into cells, a vector mainly used for replicating DNA or RNA, and a vector mainly used for expression of DNA or RNA transcription and/or translation. The vector further includes a vector with a variety of the above-described functions. The vector can be a polynucleotide that can be transcribed and translated into a polypeptide when introduced into a suitable host cell. Generally, by culturing a suitable host cell containing the vector, the vector can produce the desired expression product.

In the present application, the term “cell” generally refers to an individual cell, cell line, or cell culture that can include or has included a plasmid or vector containing the nucleic acid molecule of the present application, or can express the antibody or its antigen-binding fragment of the present application. The host cell can include the progeny of a single host cell. Due to natural, accidental or deliberate mutations, the progeny cells may not be exactly the same as the original parent cells in terms of morphology or genome, as long as they can express the antibody or its antigen-binding fragment of the present application. The host cell can be obtained by transfecting cells in vitro with the vector of the present application. The host cell can be a prokaryotic cell (e.g., Escherichia coli) or a eukaryotic cell (e.g., yeast cells, e.g., COS cells, Chinese Hamster Ovary (CHO) cells, HeLa cells, HEK293 cells, COS-1 cells, NS0 cells, or myeloma cells). In some embodiments, the host cell is a mammalian cell. For example, the mammalian cell can be a CHO-K1 cell. In the present application, the term “recombinant host cell” generally refers to a cell into which a recombinant expression vector is introduced. The recombinant host cell includes not only a certain specific cell, but also the progeny thereof.

In the present application, the term “about” refers generally to a variation within 0.5%-10% of the given value, e.g., a variation within 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the given value.

In the present application, the term “include” generally means comprise, contain, have, or include. In some cases, it also means “be” or “consist of”.

In one aspect, the present application provides an isolated antigen-binding protein, wherein the isolated antigen-binding protein can have one or more properties selected from the group consisting of: 1) an ability of specifically binding to VEGFA (Vascular Endothelial Growth Factor A); 2) an ability of preventing VEGF from binding its corresponding receptor(s); and 3) an ability of inhibiting VEGF-driven biological functions.

In some embodiments, the VEGFA comprises a human VEGFA. In some embodiments, the VEGF-driven biological function comprises angiogenesis.

In the present application, the VEGFA can include a variant of the VEGFA. For example, the variant can be: 1) a protein or polypeptide formed by substitution, deletion, or addition of one or more amino acids in the amino acid sequence of the VEGFA protein; and 2) a protein or polypeptide with at least about 85% (e.g., at least about 85%, about 90%, about 91%, about 92%, about 93%, about 94%, about 95%, about 96%, about 97%, about 98%, about 99% or higher) of sequence homology with the VEGFA protein.

In some embodiments, the isolated antigen-binding protein comprises at least one CDR (complementarity determining region) of the variable region of the heavy chain (VH) of an antibody, for example, a single domain antibody, wherein the variable region comprises any one of the amino acid sequences as set forth in SEQ ID NO: 13-16. In some embodiment, the sequence of the CDRs can be defined by any known numbering strategy, for example, Kabat, Chothia, IMGT, or combination thereof. In the present application, the CDRs amino acid sequence can be specified upon Chothia and Kabat schemes.

In the present application, the isolated antigen-binding protein can comprise a HCDR3, the HCDR3 can comprise an amino acid sequence as set forth in any one of SEQ ID NO: 1, SEQ ID NO: 4, SEQ ID NO: 7, and SEQ ID NO: 10.

In the present application, the isolated antigen-binding protein can comprise a HCDR2, the HCDR2 can comprise an amino acid sequence as set forth in any one of SEQ ID NO: 2, SEQ ID NO: 5, SEQ ID NO: 8, and SEQ ID NO: 11.

In the present application, the isolated antigen-binding protein can comprise a HCDR1, the HCDR1 can comprise an amino acid sequence as set forth in any one of SEQ ID NO: 3, SEQ ID NO: 6, SEQ ID NO: 9, and SEQ ID NO: 12.

In the present application, the isolated antigen-binding protein can comprise a HCDR1, a HCDR2, and a HCDR3, the HCDR1 can comprise an amino acid sequence as set forth in any one of SEQ ID NO: 3, SEQ ID NO: 6, SEQ ID NO: 9, and SEQ ID NO: 12, the HCDR2 can comprise an amino acid sequence as set forth in any one of SEQ ID NO: 2, SEQ ID NO: 5, SEQ ID NO: 8, and SEQ ID NO: 11, the HCDR3 can comprise an amino acid sequence as set forth in any one of SEQ ID NO: 1, SEQ ID NO: 4, SEQ ID NO: 7, and SEQ ID NO: 10.

In the present application, the isolated antigen-binding protein can comprise a HCDR1, a HCDR2, and a HCDR3, wherein the HCDR1, the HCDR2, and the HCDR3 can comprise the amino acid sequences selected any one of the groups consisting of:
1) the HCDR1 comprises an amino acid sequence as set forth in SEQ ID NO: 3, the HCDR2 comprises an amino acid sequence as set forth in SEQ ID NO: 2, and the HCDR3 comprises an amino acid sequence as set forth in SEQ ID NO: 1;
2) the HCDR1 comprises an amino acid sequence as set forth in SEQ ID NO: 6, the HCDR2 comprises an amino acid sequence as set forth in SEQ ID NO: 5, and the HCDR3 comprises an amino acid sequence as set forth in SEQ ID NO: 4;
3) the HCDR1 comprises an amino acid sequence as set forth in SEQ ID NO: 9, the HCDR2 comprises an amino acid sequence as set forth in SEQ ID NO: 8, and the HCDR3 comprises an amino acid sequence as set forth in SEQ ID NO: 7; and
4) the HCDR1 comprises an amino acid sequence as set forth in SEQ ID NO: 12, the HCDR2 comprises an amino acid sequence as set forth in SEQ ID NO: 11, and the HCDR3 comprises an amino acid sequence as set forth in SEQ ID NO: 10.

In the present application, the isolated antigen-binding protein can comprise a H-FR1, wherein a C-terminus of the H-FR1 is directly or indirectly linked to an N-terminus of the HCDR1, and the H-FR1 comprises an amino acid sequence as set forth in SEQ ID NO: 35. In some embodiments, the H-FR1 comprises an amino acid sequence as set forth in SEQ ID NO: 24 or SEQ ID NO: 28.

In the present application, the isolated antigen-binding protein can comprise a H-FR2, wherein the H-FR2 is located between the HCDR1 and the HCDR2, and the H-FR2 comprises an amino acid sequence as set forth in SEQ ID NO: 36. In some embodiments, the H-FR2 can comprises an amino acid sequence as set forth in SEQ ID NO: 25, SEQ ID NO: 29, SEQ ID NO: 31, or SEQ ID NO: 33.

In the present application, the isolated antigen-binding protein can comprise a H-FR3, wherein the H-FR3 is located between the HCDR2 and the HCDR3, and the H-FR3 comprises an amino acid sequence as set forth in SEQ ID NO: 37. In some embodiments, the H-FR3 can comprise an amino acid sequence as set forth in SEQ ID NO: 26, SEQ ID NO: 30, SEQ ID NO: 32, or SEQ ID NO: 34.

In the present application, the isolated antigen-binding protein can comprise a H-FR4, wherein an N-terminus of the H-FR4 is directly or indirectly linked to a C-terminus of the HCDR3, and the H-FR4 comprises an amino acid sequence as set forth in SEQ ID NO: 27.

In the present application, the isolated antigen-binding protein can comprise a H-FR1, a H-FR2, a H-FR3, and a H-FR4, wherein the H-FR1 can comprise an amino acid sequence as set forth in any one of SEQ ID NO: 24 and SEQ ID NO: 28, the H-FR2 can comprise an amino acid sequence as set forth in any one of SEQ ID NO: 25, SEQ ID NO: 29, SEQ ID NO: 31 and SEQ ID NO: 33, the H-FR3 can comprise an amino acid sequence as set forth in any one of SEQ ID NO: 26, SEQ ID NO: 30, SEQ ID NO: 32, and SEQ ID NO: 34, and the H-FR4 can comprise an amino acid sequence as set forth in SEQ ID NO: 27.

In the present application, the isolated antigen-binding protein can comprise a H-FR1, a H-FR2, a H-FR3, and a H-FR4, wherein the H-FR1, the H-FR2, the H-FR3 and the H-FR4 can comprise the amino acid sequences selected any one from the group consisting of:
1) the H-FR1 comprises an amino acid sequence as set forth in SEQ ID NO: 24, the H-FR2 comprises an amino acid sequence as set forth in SEQ ID NO: 25, the H-FR3 comprises an amino acid sequence as set forth in SEQ ID NO: 26, and the H-FR4 comprises an amino acid sequence as set forth in SEQ ID NO: 27;
2) the H-FR1 comprises an amino acid sequence as set forth in SEQ ID NO: 24, the H-FR2 comprises an amino acid sequence as set forth in SEQ ID NO: 31, the H-FR3 comprises an amino acid sequence as set forth in SEQ ID NO: 32, and the H-FR4 comprises an amino acid sequence as set forth in SEQ ID NO: 27;
3) the H-FR1 comprises an amino acid sequence as set forth in SEQ ID NO: 24, the H-FR2 comprises an amino acid sequence as set forth in SEQ ID NO: 33, the H-FR3 comprises an amino acid sequence as set forth in SEQ ID NO: 34, and the H-FR4 comprises an amino acid sequence as set forth in SEQ ID NO: 27; and
4) the H-FR1 comprises an amino acid sequence as set forth in SEQ ID NO: 28, the H-FR2 comprises an amino acid sequence as set forth in SEQ ID NO: 29, the H-FR3 comprises an amino acid sequence as set forth in SEQ ID NO: 30, and the H-FR4 comprises an amino acid sequence as set forth in SEQ ID NO: 27.

In the present application, the isolated antigen-binding protein can comprise a heavy chain variable region (VH), and the VH can comprise an amino acid sequence as set forth in any one of SEQ ID NO: 13-16.

In the present application, the isolated antigen-binding protein can comprise an antibody or its antigen-binding fragment.

In some embodiments, the antibody can be selected from the group consisting of a single monoclonal antibody, single strand antibody, chimeric antibody, polyspecific antibody, humanized antibody and fully human antibody.

In some embodiments, the antigen-binding fragment can be selected from the group consisting of Fab, Fab′, F(ab)₂, F(ab′)₂, sdAb, Fv and ScFv fragment or derived bi-paratopic antigen-binding protein.

In the present application, the isolated antigen-binding protein can comprise a single domain antibody.

In the present application, the single domain antibody can comprise a HCDR3, the HCDR3 can comprise an amino acid sequence as set forth in any one of SEQ ID NO: 1, SEQ ID NO: 4, SEQ ID NO: 7, and SEQ ID NO: 10.

In the present application, the single domain antibody can comprise a HCDR2, the HCDR2 can comprise an amino acid sequence as set forth in any one of SEQ ID NO: 2, SEQ ID NO: 5, SEQ ID NO: 8 and SEQ ID NO: 11.

In the present application, the single domain antibody can comprise a HCDR1, the HCDR1 can comprise an amino acid sequence as set forth in any one of SEQ ID NO: 3, SEQ ID NO: 6, SEQ ID NO: 9, and SEQ ID NO: 12.

In the present application, the single domain antibody can comprise a HCDR1, a HCDR2, and a HCDR3, the HCDR1 can comprise an amino acid sequence as set forth in any one of SEQ ID NO: 3, SEQ ID NO: 6, SEQ ID NO: 9, and SEQ ID NO: 12, the HCDR2 can comprise an amino acid sequence as set forth in any one of SEQ ID NO: 2, SEQ ID NO: 5, SEQ ID NO: 8, and SEQ ID NO: 11, the HCDR3 can comprise an amino acid sequence as set forth in any one of SEQ ID NO: 1, SEQ ID NO: 4, SEQ ID NO: 7, and SEQ ID NO: 10.

In the present application, the single domain antibody can comprise a HCDR1, a HCDR2, and a HCDR3, wherein the HCDR1, the HCDR2, and the HCDR3 can comprise the amino acid sequences selected any one of the groups consisting of:
1) the HCDR1 can comprise an amino acid sequence as set forth in SEQ ID NO: 3, the HCDR2 can comprise an amino acid sequence as set forth in SEQ ID NO: 2, and the HCDR3 can comprise an amino acid sequence as set forth in SEQ ID NO: 1;
2) the HCDR1 can comprise an amino acid sequence as set forth in SEQ ID NO: 6, the HCDR2 can comprise an amino acid sequence as set forth in SEQ ID NO: 5, and the HCDR3 can comprise an amino acid sequence as set forth in SEQ ID NO: 4;
3) the HCDR1 can comprise an amino acid sequence as set forth in SEQ ID NO: 9, the HCDR2 can comprise an amino acid sequence as set forth in SEQ ID NO: 8, and the HCDR3 can comprise an amino acid sequence as set forth in SEQ ID NO: 7; and
4) the HCDR1 can comprise an amino acid sequence as set forth in SEQ ID NO: 12, the HCDR2 can comprise an amino acid sequence as set forth in SEQ ID NO: 11, and the HCDR3 can comprise an amino acid sequence as set forth in SEQ ID NO: 10.

In the present application, the single domain antibody can comprise a H-FR1, wherein a C-terminus of the H-FR1 is directly or indirectly linked to an N-terminus of the HCDR1, and the H-FR1 can comprise an amino acid sequence as set forth in SEQ ID NO: 35. In some embodiments, the H-FR1 can comprise an amino acid sequence as set forth in SEQ ID NO: 24 or SEQ ID NO: 28.

In the present application, the single domain antibody can comprise a H-FR2, wherein the H-FR2 is located between the HCDR1 and said HCDR2, and the H-FR2 can comprise an amino acid sequence as set forth in SEQ ID NO: 36. In some embodiments, the H-FR2 can comprise an amino acid sequence as set forth in SEQ ID NO: 25, SEQ ID NO: 29, SEQ ID NO: 31, or SEQ ID NO: 33.

In the present application, the single domain antibody can comprise a H-FR3, wherein the H-FR3 is located between the HCDR2 and the HCDR3, and the H-FR3 can comprise an amino acid sequence as set forth in SEQ ID NO: 37. In some embodiments, the H-FR3 can comprise an amino acid sequence as set forth in SEQ ID NO: 26, SEQ ID NO: 30, SEQ ID NO: 32, or SEQ ID NO: 34.

In the present application, the single domain antibody can comprise a H-FR4, wherein an N-terminus of the H-FR4 is directly or indirectly linked to a C-terminus of the HCDR3, and the H-FR4 can comprise an amino acid sequence as set forth in SEQ ID NO: 27.

In the present application, the single domain antibody can comprise a H-FR1, a H-FR2, a H-FR3, and a H-FR4, wherein the H-FR1 can comprise an amino acid sequence as set forth in any one of SEQ ID NO: 24 and SEQ ID NO: 28, the H-FR2 can comprise an amino acid sequence as set forth in any one of SEQ ID NO: 25, SEQ ID NO: 29, SEQ ID NO: 31, and SEQ ID NO: 33, the H-FR3 can comprise an amino acid sequence as set forth in any one of SEQ ID NO: 26, SEQ ID NO: 30, SEQ ID NO: 32, and SEQ ID NO: 34, and the H-FR4 can comprise an amino acid sequence as set forth in SEQ ID NO: 27.

In the present application, the single domain antibody can comprise a H-FR1, a H-FR2, a H-FR3, and a H-FR4, wherein the H-FR1, the H-FR2, the H-FR3 and the H-FR4 can comprise the amino acid sequences selected any one from the group consisting of:
1) the H-FR1 can comprise an amino acid sequence as set forth in SEQ ID NO: 24, the H-FR2 can comprise an amino acid sequence as set forth in SEQ ID NO: 25, the H-FR3 can comprise an amino acid sequence as set forth in SEQ ID NO: 26, and the H-FR4 can comprise an amino acid sequence as set forth in SEQ ID NO: 27;
2) the H-FR1 can comprise an amino acid sequence as set forth in SEQ ID NO: 24, the H-FR2 can comprise an amino acid sequence as set forth in SEQ ID NO: 31, the H-FR3 can comprise an amino acid sequence as set forth in SEQ ID NO: 32, and the H-FR4 can comprise an amino acid sequence as set forth in SEQ ID NO: 27;
3) the H-FR1 can comprise an amino acid sequence as set forth in SEQ ID NO: 24, the H-FR2 can comprise an amino acid sequence as set forth in SEQ ID NO: 33, the H-FR3 can comprise an amino acid sequence as set forth in SEQ ID NO: 34, and the H-FR4 can comprise an amino acid sequence as set forth in SEQ ID NO: 27; and
4) the H-FR1 can comprise an amino acid sequence as set forth in SEQ ID NO: 28, the H-FR2 can comprise an amino acid sequence as set forth in SEQ ID NO: 29, the H-FR3 can comprise an amino acid sequence as set forth in SEQ ID NO: 30, and the H-FR4 can comprise an amino acid sequence as set forth in SEQ ID NO: 27.

In the present application, the single domain antibody can comprise a heavy chain variable region (VHH), and the VHH can comprise an amino acid sequence as set forth in any one of SEQ ID NO: 13-16.

In another aspect, the present application provides a bi-paratopic antigen-binding protein, wherein the bi-paratopic antigen-binding protein can comprise a first antigen-binding domain, and a second binding domain. In some embodiments, the first antigen-binding protein and the second antigen-binding domain can be linked via a linker. In some embodiments, the first antigen-binding protein and the second antigen-binding protein can be linked without a linker.

In the present application, the first antigen-binding domain of the bi-paratopic antigen-binding protein can comprise the isolated antigen-binding protein of the present application. In the present application, the second antigen-binding domain of the bi-paratopic antigen-binding protein can comprise the isolated antigen-binding protein of the present application. In some embodiments, the first antigen-binding domain and the second antigen-binding domain of the bi-paratopic antigen-binding protein can comprise the isolated antigen-binding protein of the present application.

In the present application, the first antigen-binding domain and the second antigen-binding domain of the bi-paratopic antigen-binding protein can target the same antigen.

In the present application, the first antigen-binding domain and the second antigen-binding domain of the bi-paratopic antigen-binding protein can bind the different epitopes.

In the present application, the first antigen-binding domain and the second antigen-binding domain of the bi-paratopic antigen-binding protein can have the different amino acid sequences.

In some embodiments, the bi-paratopic antigen-binding protein can comprise CDRs amino acid sequences selected from the group consisting of: 1) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 6, the HCDR2 comprising the amino acid sequence as set forth in SEQ ID NO: 5, the HCDR3 comprising the amino acid sequence as set forth in SEQ ID NO: 4; 2) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 9, the HCDR2 comprising the amino acid sequence as set forth in SEQ ID NO: 8, the HCDR3 comprising the amino acid sequence as set forth in SEQ ID NO: 7; and 3) First antigen binding domain: the HCDR1 comprising the amino acid sequence as set forth in SEQ ID NO: 3, the HCDR2 comprising the amino acid sequence as set forth in SEQ ID NO: 2, the HCDR3 comprising the amino acid sequence as set forth in SEQ ID NO: 1; Second antigen binding domain: the HCDR1 comprising the amino acid sequence as set forth in SEQ ID NO: 12, the HCDR2 comprising the amino acid sequence as set forth in SEQ ID NO: 11, the HCDR3 comprising the amino acid sequence as set forth in SEQ ID NO: 10.

In some embodiments, the bi-paratopic antigen-binding protein can comprise VHH sequences selected from any one of the groups consisting of:
1) the first antigen-binding protein can comprise a VHH amino acid sequence as set forth in SEQ ID NO: 13; the second antigen-binding protein can comprise a VHH amino acid sequence as set forth in SEQ ID NO: 14;
2) the first antigen-binding protein can comprise a VHH amino acid sequence as set forth in SEQ ID NO: 13; the second antigen-binding protein can comprise a VHH amino acid sequence as set forth in SEQ ID NO: 15; and
3) the first antigen-binding protein can comprise a VHH amino acid sequence as set forth in SEQ ID NO: 13; the second antigen-binding protein can comprise a VHH amino acid sequence as set forth in SEQ ID NO: 16.

In the present application, the bi-paratopic antigen-binding protein can comprise an amino acid sequence as set forth in any one of SEQ ID NO: 17-19.

In another aspect, the present application provides a polypeptide, wherein the polypeptide can comprise the isolated binding protein, and other functional domain.

In another aspect, the present application provides a polypeptide, wherein the polypeptide can comprise the bi-paratopic antigen-binding protein, and other functional domain.

In some embodiments, the functional domain can comprise a Fc region. In some embodiments, the Fc region can comprise a human Fc region. In some embodiments, the Fc region can comprise a human IgG Fc region or its variants, for example, the Fc region can comprise at least about 85% (e.g., at least about 85%, about 90%, about 91%, about 92%, about 93%, about 94%, about 95%, about 96%, about 97%, about 98%, about 99% or higher) of sequence homology with the wild-type human IgG Fc region. In some embodiments, the Fc region can comprise an amino acid sequence as set forth in SEQ ID NO: 20 or with at least about 85% of sequence homology.

In the present application, the functional domain can comprise a complement-inhibiting entity. For example, the complement-inhibiting entity can comprise a complement H (CFH) fragment. For example, the complement H (CFH) fragment can comprise a human complement H (CFH) fragment. For example, the complement H (CFH) fragment can comprise an amino acid sequence as set forth in SEQ ID NO: 22.

In some embodiments, the polypeptide can comprise an amino acid sequence as set forth in SEQ ID NO: 21 or SEQ ID NO: 23.

In some embodiments, the antigen-binding protein SLN6068 comprises an amino acid sequence of SEQ ID NO: 13. In some embodiments, the antigen-binding protein SLN6043 comprises an amino acid sequence of SEQ ID NO: 14. In some embodiments, the antigen-binding protein SLN6062 comprises an amino acid sequence of SEQ ID NO: 15. In some embodiments, the antigen-binding protein SLN6065 comprises an amino acid sequence of SEQ ID NO: 16. In some embodiments, the antigen-binding protein SLN6071 comprises an amino acid sequence of SEQ ID NO: 17. In some embodiments, the antigen-binding protein SLN6075 comprises an amino acid sequence of SEQ ID NO: 18. In some embodiments, the antigen-binding protein SLN6079 comprises an amino acid sequence of SEQ ID NO: 19. In some embodiments, the antigen-binding protein SLN6073 comprises an amino acid sequence of SEQ ID NO: 21. In some embodiments, the antigen-binding protein SLN6074 comprises an amino acid sequence of SEQ ID NO: 23.

In the present application, the antigen-binding protein can be isolated or purified.

In another aspect, the present application provides one or more isolated nucleic acid molecules, wherein the nucleic acid molecules encode the isolated antigen-binding protein, the bi-paratopic antigen-binding protein or the polypeptide. In the present application, the nucleic acid encoding the isolated antigen-binding protein, the bi-paratopic antigen-binding protein or the polypeptide can be prepared by various methods known in the art, including, but not limited to, overlapping PCR by using restrictive fragment operation or using synthetic oligonucleotide. See, e.g., Sambrook et al., Molecular Cloning, A Laboratory Manual, Cold Spring Harbor Laboratory Press, Cold Spring Harbor, N.Y., 1989; and Ausube et al. Current Protocols in Molecular Biology, Greene Publishing and Wiley-Interscience, New York N.Y., 1993.

In another aspect, the present application provides one or more vectors including the one or more nucleic acid molecules of the present application. Each vector can include one or more nucleic acid molecules. Moreover, the vector can further include other gene(s), e.g., a marker gene that allows the selection of the vector in an appropriate host cell and under appropriate conditions. Moreover, the vector can further include an expression control element that allows the coding region to be properly expressed in an appropriate host. Such control element is well known by persons skilled in the art, e.g., it can include promoter, ribosome binding site, enhancer and other control elements regulating the transcription of gene or translation of mRNA, and the like. The one or more nucleic acid molecules of the present application can be operatively linked to the expression control element.

The vector can include, e.g., plasmid, cosmid, virus, phage, or other vectors commonly used in, e.g., genetic engineering. For example, the vector is an expression vector.

In another aspect, the present application provides a cell, and the cell can include the one or more nucleic acid molecules of the present application and/or the one or more vectors of the present application. For example, each cell can include one nucleic acid molecule or one vector of the present application. For example, each cell or each kind of cell can include a plurality of (e.g., two or more, e.g., two kinds or more kinds) nucleic acid molecules or vectors of the present application. For example, the vector of the present application can be introduced into cells, e.g., eukaryotic cells, such as cells from plants, fungi or yeast cells, etc. The vectors of the present application can be introduced into cells by methods known in the art, such as electroporation, lipofectine transfection, lipofectamin transfection, and the like.

In another aspect, the present application provides a method of preparing the isolated antigen-binding protein, the bi-paratopic antigen-binding protein or the polypeptide. The method can include culturing the host cell of the present application under conditions that allow the antibody or its antigen-binding fragment to be expressed. For example, the method can include using an appropriate medium, an appropriate temperature, and culturing time, that are understood by persons of ordinary skills in the art.

In another aspect, the present application provides a pharmaceutical composition comprising the isolated antigen-binding protein, the bi-paratopic antigen-binding protein or the polypeptide, and optionally pharmaceutically acceptable adjuvants.

The pharmaceutically acceptable adjuvants can include buffers, antioxidants, preservatives, low molecular weight polypeptides, proteins, hydrophilic polymers, amino acids, carbohydrates, chelating agents, counterions, metal complexes and/or nonionic surfactants, etc.

In the present application, the pharmaceutical composition can be formulated for oral administration, intravenous administration, intramuscular administration, in situ administration at the tumor site, inhalation, rectal administration, vaginal administration, transdermal administration or administration via subcutaneous depot.

In another aspect, the present application provides use of the isolated antigen-binding protein, the bi-paratopic antigen-binding protein, the polypeptide the nucleic acid molecules, the vectors, the cell, and/or the pharmaceutical composition in preparation of a drug, wherein the drug is used for preventing or treating a disease.

In another aspect, the present application provides the isolated antigen-binding protein, the bi-paratopic antigen-binding protein, the polypeptide the nucleic acid molecules, the vectors, the cell, and/or the pharmaceutical composition, for use in preventing or treating a disease.

In another aspect, the present application provides a method of preventing or treating a disease in a subject in need thereof, including administering to the subject the isolated antigen-binding protein, the bi-paratopic antigen-binding protein, the polypeptide the nucleic acid molecules, the vectors, the cell, and/or the pharmaceutical composition.

In the present application, the disease can comprise a VEGFA-associated disease. For example, the disease can comprise a tumor. For example, the disease can comprise an age-related macular degeneration. For example, the disease can comprise a VEGFA-driven pathogenic process.

### EXAMPLES

The following examples are set forth so as to provide those of ordinary skill in the art with a complete disclosure and description of how to make and use the present invention, also are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Discovery of VEGF-Binding VHHs

### 1) VHH Immune Library Construction

Immunization was performed using recombinant human VEGFA165 (UniProt identifier P15692-4) in two healthy camels. On day 90 after finishing 6 rounds of immunization, Camel serum was collected and used to measure titers of the antigen-specific antibodies.

Briefly, ELISA analysis was performed to measure the antibody titer in camel serum. Specially, human VEGFA165 was diluted to a final concentration as of 5 μg/ml in Phosphate-buffered saline (PBS), 100 μl of the solution was used for coating in each well of a 96-well ELISA microplate and incubate at room temperature (RT) for 1 hour (hr). Each well was washed three times with 300 μl washing buffer (0.05% Tween-20 in PBS, or PBST) to remove unbound human VEGFA165, and then 200 μl of blocking buffer (2% BSA in PBS) were added to each well and incubated at room temperature for 2 hr at RT. A test serum sample was serially 2-fold diluted to make 15 test samples from 1/100 to 1/1638400. After blocking, each well was washed with 300 μl washing buffer for three times and then each of the serially diluted test sample was added to each well and incubate at RT for 1 hr. After three times washing, 100 μl of 1:10000 diluted secondary antibody conjugated to horseradish peroxidase (HRP-Goat anti-Llama IgG(H+L), Novex cat #A16060) was added to each well and incubate at RM for 1 hr. After three times of washing again, 100 μl of TMB solution were added to each well and allowed to react at RT for 10 mins. After adding 100 μl of stop solution (2 mol/L (M) HCl), the microplates were measured at OD450 nm.

To construct a phage-displayed VHH library, 200 ml of blood was collected from each of the immunized camels and PBMCs were recovered for VHH immune library construction by following a standardized protocol. The final phage-displayed VHH library had 3.4×10⁹ independent clones, with 91% of them encoding VHH-gp3 fusion proteins.

### 2) Phage Panning Method

For Round 1 panning, 300 μl (6.0×10¹³ CFU) phages was incubated with 10 g of biotinylated recombinant human VEGFA121-Avi-His tag or mouse VEGFA120-Avi-His tag protein within 1 ml of blocking buffer (1% BSA in PBS) at RT for 1 hr to make phages/target mixture. At the same time, 100 μL of Streptavidin-coated Dynabeads M-280 (Invitrogen, 11206D) were washed with 1 mL of blocking buffer for five times in an Eppendorf microtube. Thereafter, the phage/target mixture was incubated with the Mag-beads prepared as above on a rotator at RT for 30 min. To recover the phages binding to the Mag-beads, the reaction tube was placed on a magnetic rack for 30 s. After the supernatant was removed, the beads were washed with 1 mL of PBST for 10 times, followed by one-time washing with PBS. The phages were eluted by adding 1 ml of trypsin (10 μg/ml in PBS) at 37° C. for 30 min.

To amplify the eluted phages, the eluted phages were added into a 15-ml tube containing 4 ml of E. Coli TG1 culture at exponential phase (A600≈0.6) and incubated without shaking at 37° C. for 30 min. After the infection, the bacterial culture was centrifuged at 4,000 rpm for 20 min at 4° C. and resuspended in 500 μl of 2×YT medium for spreading onto 2×YT-GA plates containing 2% Glucose and 100 μg/ml Ampicillin in 2×YT, and incubated overnight at 37° C. To amplify phages, collect the bacteria on the second day and inoculate with 100 ml of 2×YT-GA at 37° C. with shaking at 250 rpm to reach a cell density at an A600 at 0.6 (approximately 1-2 h). The phages were rescued by adding helper phage M13K07 at a phage: bacteria ratio of 1000. After Incubating the culture at 37° C. without shaking for 30 min, the culture was continued for 30 min with shaking at 180 rpm. Medium was changed to 2×YT-AK (Amp:100 μg/ml, Kan:50 μg/ml) before the culture was placed back to incubator for shaking at 250 rpm at 30° C. for 4 hrs.

Following amplification, the bacterial pellets was removed by centrifugation for 20 min at 6000 rpm at 4° C., and the phage were precipitated from the supernatant by adding ¼ volume of PEG solution (20% Polyethylene glycol 6000, 2.5M NaCl) and staying on ice overnight. After centrifugation at 10,000 rpm for 30 min, the pellets were resuspended in 5 ml of PBS, insoluble debris were removed by centrifugation at 8000 rpm for 10 min at 4° C. PEG precipitation was repeated once as described above. The final phage pellets were resuspended in 1 ml of PBS, and tittered to be used as input for the next round of selection. Round 2 and 3 were performed as described for Round 1, the variation was the decreased amount of the antigen or antigens derived from different species to have cross-reactive phage clones. Panning summary was listed in table1.

TABLE 1
Panning summary

| Panning round | Library | input (CFU) | output (CFU) | Ratio(I/O) |
|---|---|---|---|---|
| 1^{st} (10 ug) | 1904A-H-1st | 3 × 10¹² | 1 × 10⁷ | 3 × 10⁵ |
| | 1904A-M-1st | 3 × 10¹² | 1 × 10⁷ | 3 × 10⁵ |
| 2^{nd} (1.5 ug) | 1904A-H-H-2nd | 7 × 10¹² | 3 × 10⁷ | 2.3 × 10⁵ |
| | 1904A-H-M-2nd | 7 × 10¹² | 2 × 10⁷ | 3.5 × 10⁵ |
| | 1904A-M-H-2nd | 7 × 10¹² | 13 × 10⁷ | 0.5 × 10⁵ |
| | 1904A-M-M-2nd | 7 × 10¹² | 2.5 × 10⁷ | 2.8 × 10⁵ |
| 3^{rd} (0.5 ug) | 1904A-H-H-H-3rd | 8 × 10¹² | 5 × 10⁸ | 1.6 × 10⁴ |
| | 1904A-H-M-M-3rd | 3 × 10¹² | 3 × 10⁸ | 1 × 10⁴ |
| | 1904A-M-H-H-3rd | 8 × 10¹² | 2 × 10⁸ | 4 × 10⁴ |
| | 1904A-M-M-M-3rd | 10 × 10¹² | 13 × 10⁸ | 0.76 × 10⁴ |

3) Primary Screening of Panning Outputs with Phage ELISA

Individual bacterial colonies were picked and inoculated into 200 μl 2×YT-GA medium, cultured at 37° C. with shaking (250 rpm) for 4-5 hr. Then 10 μl of culture was transferred into a new deep 96-well plate containing 200 μl of 2×YT-GA and incubated as above till OD600 reached around 0.5. M13K07 helper phages were added to a final concentration of 1×10¹⁰ cfu, and plates were incubated at 37° C. for 30 min without shaking. After the bacteria were harvested by centrifugation at 4000 rpm for 20 min and resuspended in 350 μl of 2×YT-AK medium, the bacterial culture was kept incubation at 30° C. overnight with shaking (800 rpm). After the overnight culture was spun at 4000 rpm at 4° C. for 30 min, the supernatants were collected for phage ELISA.

For phage ELISA, immunoplates were coated with 100 μl of 1 μg/ml streptavidin per well and incubate at 4° C. overnight. After washing with PBST for 3 times and blocking with 200 μl of 1% BSA/PBS at RT for 1 hr, the recombinant hVEGFA121-biotin or mVEGFA120-biotin (0.1 μg/ml) was added at 100 μl per well and incubated at RT for 1 hr. Plates were washed with PBST for 3 times and 50 μl phage supernatant and 50 μl 1% BSA/PBST were mixed and added into each well, incubated at RT for 1 hr. Plates were washed with PBST for 3 times and 100 μl of goat anti-M13-HRP (SinoBio, 11973-MM05T-H, diluted at 1/5000 in 10% BSA/PBST) were added into each well and incubated at RT for 1 hr. Plates were then washed as before and 100 μl of TMB substrate solution were added and incubated at RT for 15 min. 100 μl/well of stop solution were added to stop the reaction before the plates were scanned with a microplate reader at 450 nm. Target-binding phages were subjected to DNA sequencing to determine the encoded VHH identities, and phages with different amino acid sequences of VHH were considered as unique clones. In total, 139 unique clones with different CDR sequences were identified as positive in target-binding assays with phage ELISA. After secondary ELISA assays by using periplasmic extractions (PPE) from the E. coli culture, part of them were selected for production of recombinant VHH-Fc proteins. Summary of screening results was listed in table2.

TABLE 2
summary of primary screening

| | | H VEGFA121- | H/M VEGFA- | total | ratio | unique | Accumulative |
|---|---|---|---|---|---|---|---|
| Screening | Library | binder (Only) | binder | binder | (binder/96) | sequence | number |
| 5.18-1^{st} | 1904A-H-H-H-3rd | 84 | 8 | 92 | 96% | 24 | 53 |
| | 1904A-M-H-H-3rd | 16 | 3 | 19 | 20% | 23 | |
| | 1904A-H-M-M-3rd | 7 | 10 | 17 | 18% | 6 | |
| | 1904A-M-M-M-3rd | 7 | 5 | 12 | 13% | 0 | |
| 5.26-2^{nd} | HMM-3rd-Plate1 | 0 | 16 | 16 | 17% | 3 | 32 |
| | HMM-3rd-Plate2 | 0 | 16 | 16 | 17% | 5 | |
| | HMM-3rd-Plate3 | 0 | 12 | 12 | 13% | 6 | |
| | HMM-3rd-Plate4 | 1 | 19 | 20 | 21% | 6 | |
| | HMM-3rd-Plate5 | 0 | 12 | 12 | 13% | 4 | |
| | HMM-3rd-Plate6 | 0 | 18 | 18 | 19% | 8 | |
| 6.03-3^{rd} | HMM-3rd-Plate7 | | | 16 | 17% | 4 | 20 |
| | HMM-3rd-Plate8 | | | 12 | 13% | 2 | |
| | HMM-3rd-Plate9 | | | 14 | 15% | 1 | |
| | HMM-3rd-Plate10 | | | 13 | 14% | 6 | |
| | HMM-3rd-Plate11 | | | 19 | 20% | 4 | |
| | HMM-3rd-Plate12 | | | 16 | 17% | 3 | |
| 6.18-4^{th} | HH-2nd-Plate13 | 10 | 4 | 14 | 15% | 8 | 20 |
| | HH-2nd-Plate14 | 5 | 1 | 6 | 6% | 3 | |
| | HH-2nd-Plate15 | 9 | 3 | 12 | 13% | 5 | |
| | HH-2nd-Plate16 | 8 | 2 | 10 | 10% | 4 | |
| 7.8-5^{th} | HH-2nd-Plate17 | 7 | 2 | 9 | 9% | 3 | 14 |
| | HH-2nd-Plate18 | 7 | 1 | 8 | 8% | 0 | |
| | HH-2nd-Plate19 | 10 | 3 | 13 | 14% | 1 | |
| | HH-2nd-Plate20 | 2 | 0 | 2 | 2% | 1 | |
| | HH-2nd-Plate21 | 2 | 0 | 2 | 2% | 1 | |
| | HH-2nd-Plate22 | 2 | 1 | 3 | 3% | 3 | |
| | HH-2nd-Plate23 | 7 | 1 | 8 | 8% | 1 | |
| | HH-2nd-Plate24 | 8 | 2 | 10 | 10% | 4 | |

| | | |
|---|---|---|
| Total | 139 | 139 |

### 4) Production and Purification of Recombinant VHH-Fc Proteins

VHH domain of selected clones were amplified by using degenerated primers (Fw: 5′ (C/g)A(g/T)gTgCAgCTggTggAgTCTgg, Rv: 5′ TgAggAgAC(A/g)gTgACC(A/T)g) to create constructs for expressing recombinant VHH-hIgG4 Fc fusion proteins within HEK293 cells. After the DNA sequences were verified with DNA sequencing, the recombinant plasmids were prepared by following standard protocols and filtered through a 0.22-μm filter before use for transfection of HEK293 cells.

To express the recombinant VHH-Fc proteins, 100 ml of Expi293F™ Cells in OPM-CD05 Medium (OPM, cat #81075-001) were cultured to reach a cell density of approximately 3˜5×10⁶ viable cells/mL with viability more than 95%. Plasmids were diluted with OPM-CD05 Medium to a concentration of 1.5 μg/ml in a total volume of 5 ml. Transfection reagent PEI (Polysciences, cat #24765-1) was diluted with OPM-CD05 Medium to a same volume of 5 ml to have a DNA:PEI ratio as 1:6 (m/m) when the diluted DNA and PEI were mixed together. After being incubated at RT for 15 minutes, the DNA/PEI complex were added onto the prepared Expi293F™ cells by swirling gently. Then the cells cultures were placed in a 37° C. incubator with ≥80% relative humidity and 5% CO₂ on an orbital shake. At 24 hr post the transfection, 5% prepton (1 mg/ml) and 2% glucose (330 g/L) were added to the culture slowly. After days of culturing, the cell culture supernatant was collected by sequential centrifugations at 1200 rpm for 10 min and 3900 rpm for 20 min before being used for Protein A purification.

VHH-Fcs were purified with Protein A Focurose 4FF (BIOON, HZ1011-2). Briefly, 1.5 ml of Protein A slurry were loaded onto a 20-ml column (G-bios, C006197-0025). After the columns were equilibrated with PBS of 10-fold of CV (column volume), the cell culture supernatant prepared as above were loaded and flow throw the Protein-A columns by gravity for 2 times. After the columns were washed with PBS for 10 times of CV, 2 ml of 0.1 M Glycine-HCl buffer (pH3.0) were used to elute the VHH-Fc proteins. The eluted proteins were neutralized with 100 ul of 1 M (pH 8.5) Tris-HCL buffer. The Protein A affinity column was regenerated and preserved by washing with PBS, ddH2O and 20% ethanol sequentially. For the eluted protein, it was desalted through an Amicon UltraCel 30K centrifugal device (Milipore, UFC903016). Briefly, eluted protein was diluted in 10 ml PBS and concentrated to 1.5 ml by centrifugation for 3 times. The final protein solution was formulated in PBS to less than 1 ml and filtrated with 0.22-μm filters.

Purity of VHH-Fcs were analyzed with SDS-PAGE. Briefly, 2 μg protein in 4×LDS Sample buffer (Genscript, M00676-10) was loaded and analyzed with SurePAGE gel (Genscript, M00653) in Tris-MOPS SDS buffer (Genscript, M00138) at a constant voltage of 160-V for 50 min. Proteins were visualized with Coomassie stain (TIANGEN, cat #PA101) following the manufacturer's instructions. The results were shown as in FIG. 1.

### 5) Characterization of VHH-Fcs

For binding ELISA, immunoplates were coated with 100 μl/well 1 μg/ml streptavidin and incubate at 4° C. overnight. Wells were washed with PBST for 3 times and blocked with 200 μl of 1% BSA/PBS at RT for 1 hr. Washed with PBST for 3 times and add hVEGFA121-biotin or mVEGFA120-biotin (0.1 μg/ml) 100 μl/well and incubated at RT for 1 hr. Plates were washed with PBST for 3 times, 100 μl/well 5-fold serially diluted VHH-Fcs from 2 μg/ml was added. and incubate at RT for 1 hr. Plates were washed with PBST for 3 times and add 100 μl goat anti-human Fc-HRP (Sigma, A0170) diluted 1/5000 in 1% BSA/PBST to each well and incubate at RT for 1 hr. Plates were then washed as before and add 100 μl TMB substrate and incubate at RT for 15 min. 100 μl per well stop solution was added to stop the reaction, and the plates were read with microplate reader at 450 nm.

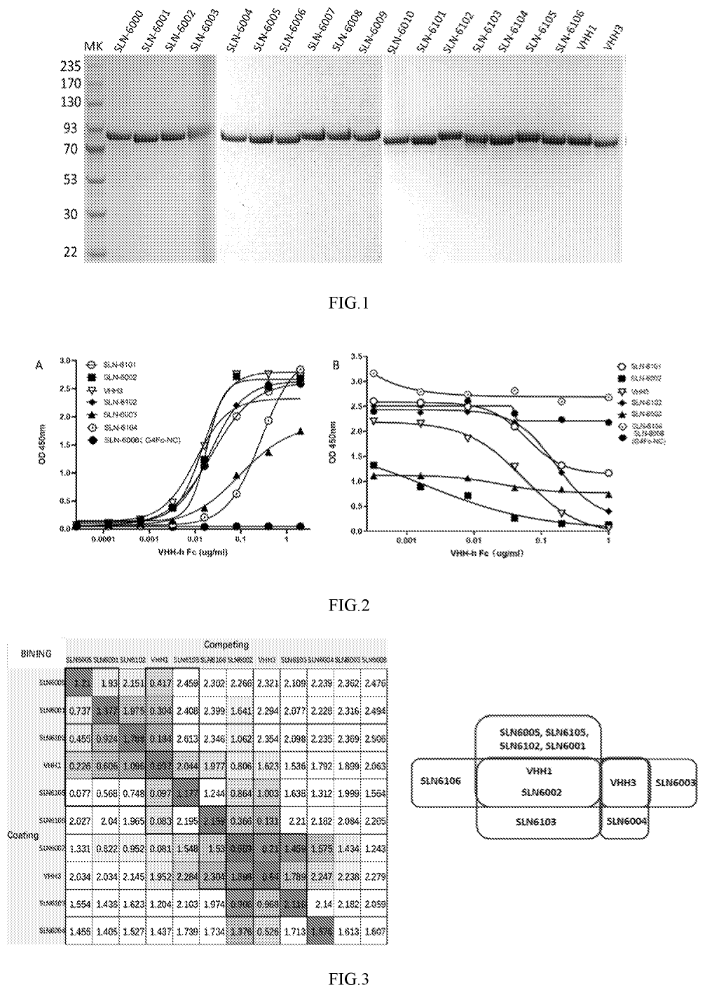

For receptor blocking assay, refer to the method above. The difference was the serially diluted VHH-Fc was pre-mixed with hVEGFA-R2-Mouse Fc (0.1 μg/ml) before being added into each well, and the secondary antibody was Goat anti-mouse IgG-Fc HRP (Abcam, ab98717). VHH-Fc binding to hVEGFA121 and receptor blocking activity was show in FIG. 2. C8, F6, C2, E7 and B3 were the top 5 strongest binders to hVEGFA121.

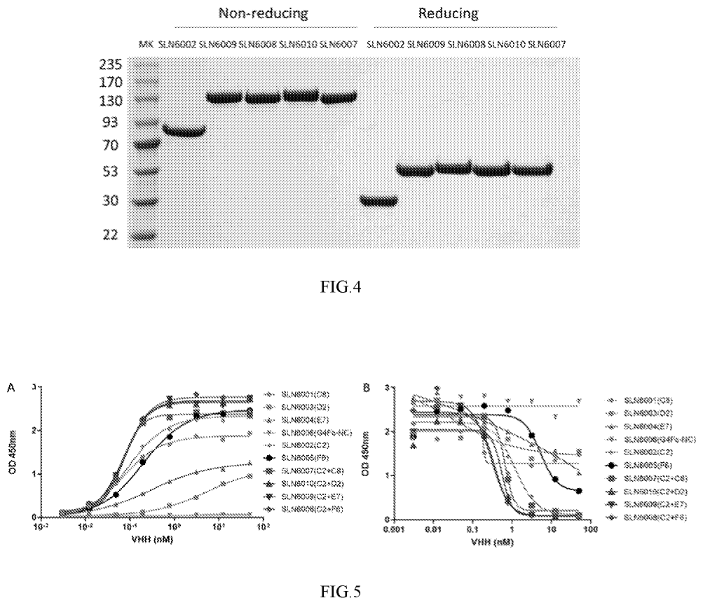

For epitope binning assay, immunoplates were coated with 100 μl/well of 5 μg/ml VHH Fc fusion protein and incubate at 4° C. overnight. Wells were washed with PBST for 3 times and blocked with 200 μl of 1% BSA/PBS at RT for 1 hr. 60 μl hVEGFA121-biotin (2 μg/ml) and 60 μl VHH-Fc fusion protein (5 μg/ml) were pre-mixed and transfer 100 μl to each well that had been coated with VHH-Fc and blocked with BSA, and continued incubation at RT for 1 hr. Plates were washed with PBST for 3 times and add 100 μl SA-HRP (Sigma, S5512) diluted 1/5000 in 1% BSA/PBST to each well and incubate at RT for 1 hr. Plates were then washed as before and add 100 μl TMB substrate and incubate at RT for 15 min. 100 μl/well of stop solution was added to stop the reaction, and the plates were read with microplate reader at 450 nm. Results was show in FIG. 3. VHHs with competitive target binding capabilities were grouped to a same bin. The results indicated F6, C8, H7 and VHH1 belong to Bin #1, C2, VHH3 and A10 belong to Bin #2, D2 might have different epitope with most of other VHH-Fc.

### 6) Bi/Tri-Specific VHH-Fc Production and Characterization

VHHs from different bins were combined with G4S linker to make Bi/Tri-paratopic VHH-Fc fusion proteins. pSLN7000 vector was used as above. Plasmid construction and protein purification can refer to the above. Production results was listed in Table3. SDS-PAGE analysis and characterization result show in FIG. 4 and FIG. 5 respectively. In both of the target-binding and receptor-blocking assays, the bi-paratopic VHHs demonstrated much better activities than those of single VHHs. among them, C2-D2, C2-F6 and C2-C8 had superior activities and thus selected as basis for further optimization. Inclusion of three non-competitive VHHs in one construct (tri-paratopics) did not improve such activities further, therefore the bi-paratopic VHHs were selected as therapeutic proteins.

TABLE 3
Summary of Bi/Tri-paratopic VHH-Fcs

| | | Conc. | Vol. | Total yield |
|---|---|---|---|---|
| Pro. Name | No. | (mg/ml) | (ml) | (mg) |
| C2 + C8 | SLN6001 | 0.80 | 1.5 | 1.2047 |
| C2 + D2 | SLN6004 | 0.75 | 1.5 | 1.124 |
| C2 + E7 | SLN6003 | 0.58 | 1.5 | 0.8752 |
| C2 + F6 | SLN6002 | 0.45 | 1.5 | 0.6712 |
| D2 + C2 + C8 | SLN6051 | 0.88 | 0.5 | 0.44 |
| F6 + C2 + D2 | SLN6052 | 0.78 | 1.5 | 1.17 |
| E7 + C2 + D2 | SLN6053 | 0.75 | 0.4 | 0.3 |
| D2 + C2 + F6 | SLN6054 | 0.74 | 1.5 | 1.11 |
| C2 + D2 + F6 | SLN6055 | 0.9 | 1.5 | 1.36 |
| C2 + C8 + D2 | SLN6056 | 0.59 | 1.5 | 0.89 |
| C2 + D2 + E7 | SLN6057 | 0.59 | 1.5 | 0.88 |
| C2 + F6 + D2 | SLN6058 | 1.2 | 1.5 | 1.8 |
| C2 + F6 + E7 | SLN6059 | 0.58 | 1.5 | 0.88 |
| F6 + C2 + E7 | SLN6060 | 0.47 | 0.5 | 0.23 |
| Aflibercept | SLN6066 | 8.02 | 1.2 | 9.63 |

### 7) Humanization of VHH-Fc Leads and Characterization

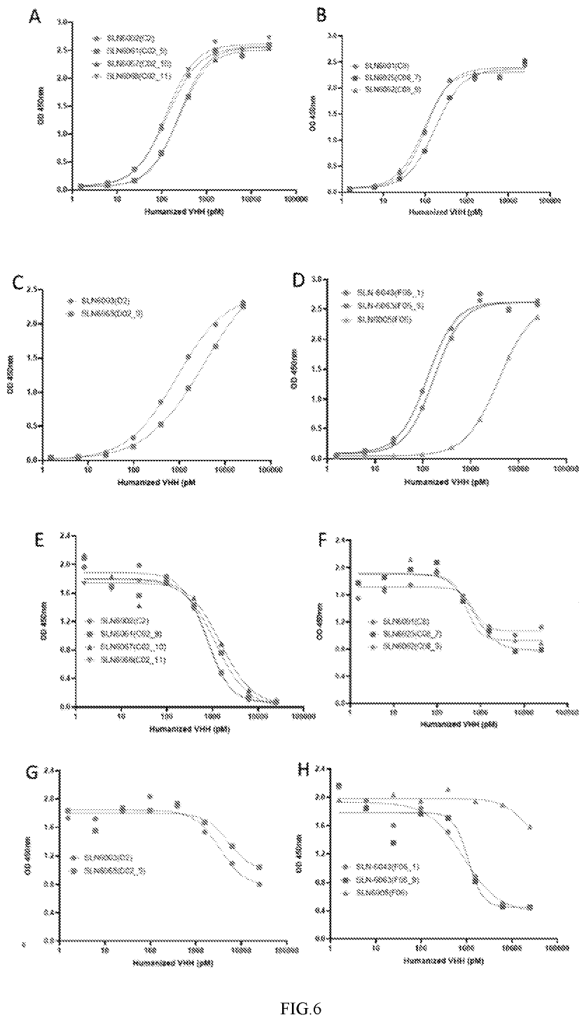

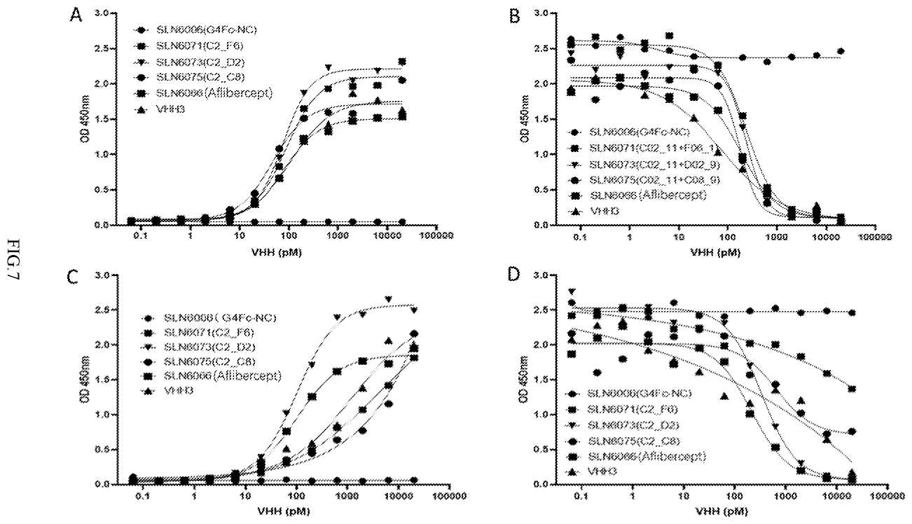

Four VHHs (C2, C8, F6, D2) were selected for humanization. Proceed with plasmid construction and protein production as above, the humanized VHH-Fc was characterized with target-binding (ELISA) and RBA. The result shown in FIG. 6 indicated that C2-11 (SLN-6068, SEQ ID NO: 13), C8-9 (SLN6062, SEQ ID NO: 15), F6-1 (SLN-6043, SEQ ID NO: 14) and D2-9 (SLN6065, SEQ ID NO: 16) sustained target-binding and receptor-blocking activities as those of the parental VHH's, and thus selected as humanized sequences for further development.

8) Bi-Paratopic VHH-Fcs with Humanized Sequences

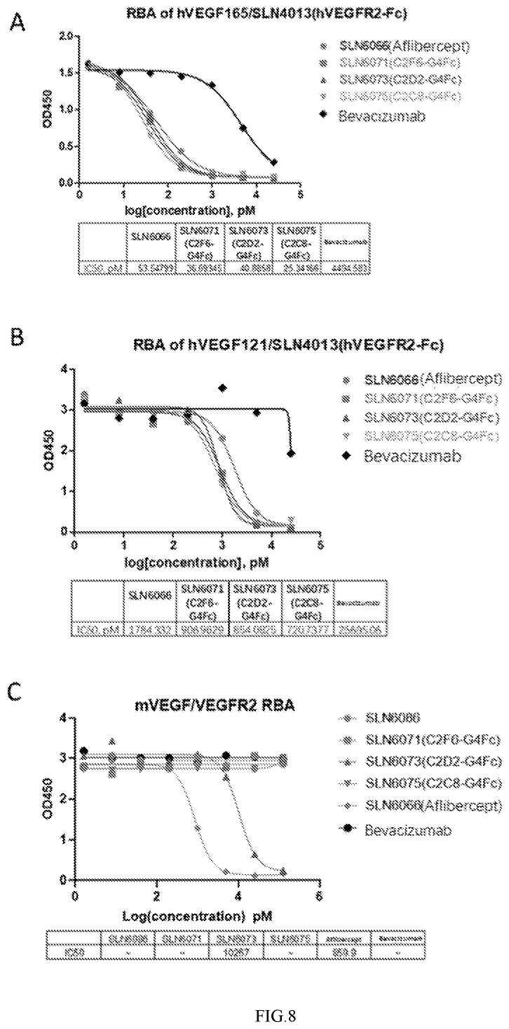

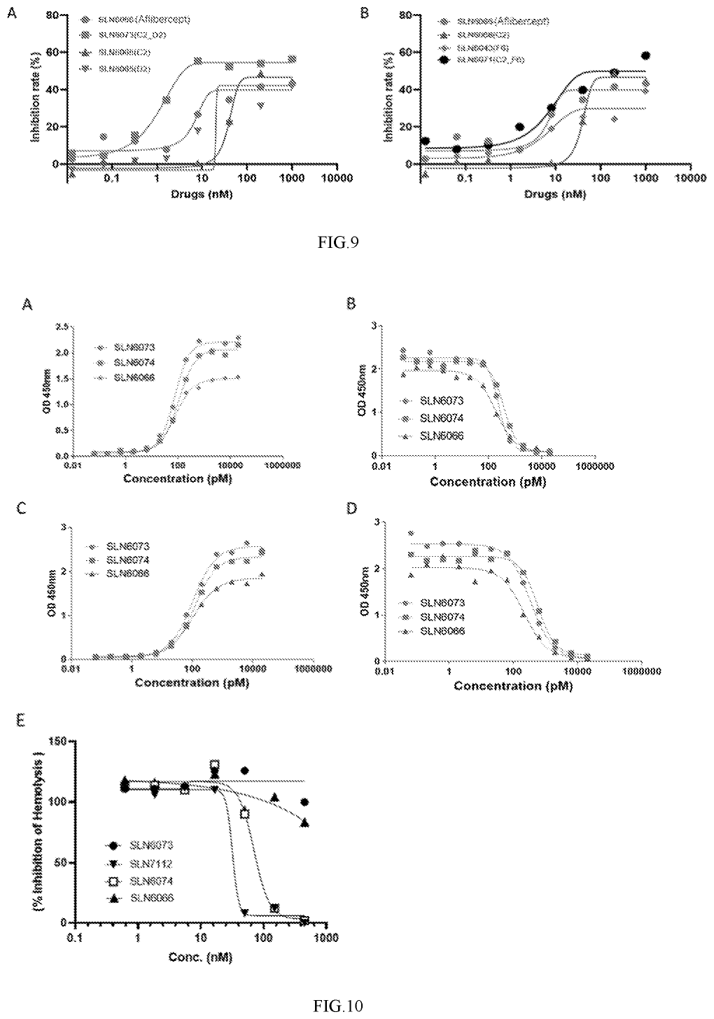

C2-11, F6-1, D2-9 and C8-9 were used for creation of bi-paratopic VHHs with humanized sequences through a G4S linker, by following procedures as described above. Three humanized bi-paratopic VHH-Fc fusion proteins were generated, named as SLN6071 (SEQ ID NO: 17), SLN6079 (SEQ ID NO: 19) and SLN6075 (SEQ ID NO: 18), the Fc region comprises an amino acid sequence of SEQ ID NO: 20. Target-binding ELISA and RBA shown in FIG. 7 indicated that such humanized bi-paratopic VHHs had biological activities comparable to that of Aflibercept, a VEGF antagonist approved by FDA for treatment of VEGF-driven angiogenesis.

### Example 2 RBA Assays of hVEGF or mVEGF on Human VEGFR2

Materials: The humanized bi-paratopic VHH-Fc fusion protein (SLN6071-6076, aflibercept (SLN6066, SEQ ID NO: 38), all made in house); bevacizumab (R & D, MAB9947-SP25 ug), hVEGFR2-mFc (made in house); hVEGFA (SLN4007, made in house), mVEGFA(SLN4011, made in house); Streptavidin (Sigma, CAT #85878); Goat-Anti-hFc-HRP (Sigma, CAT #A0170); Goat-Anti-mFc-HRP (Abcam, CAT #ab98717); TMB and stop solution (abcam, CAT #ab210902 and ab210900); Coating Buffer: 1×PBS; Washing buffer:1×PBS+0.05% Tween20; Blocking buffer:1×PBS+0.05% Tween20+1% BSA.

### Procedure:

1) Coat plates with 100 ul/well of 1 ug/ml streptavidin and incubate at 4° C. overnight. 2) Wash the plates with PBST 3 times. 3) Block the plates with 1% BSA in PBST at RT for 1 hr. 4) Add hVEGFA-biotin (0.07 ug/ml) or mVEGFA120-biotin (0.07 ug/ml) 100 ul/well and incubate at RT for 1 hr. 5) Wash the plates with PBST 3 times. 6) Prepare serial dilutions of the test articles (the humanized bi-paratopic VHH-VHH-Fcs, aflibercept, bevacizumab) starting from 25000 pM, before adding 100 ul of the serially diluted articles to the microplate wells for incubation at RT for 1 hr. 7) Wash the plate wells with PBST for 3 times before adding 100 ul of hVEGFR2 (0.14 ug/ml) and incubating at RT for 1 hr. 8) Wash the plate wells with PBST for 3 times before incubating with goat anti-human Fc-HRP (1:5000) at RT for 1 hr. 9) Wash the plate wells with PBST 3 times before adding 100 ul/well of TMB for incubation at RT for 15 min. 10). Quench with 100 ul/well stop solution before scanning the plates with microplate reader at 450 nm.

The result is shown in FIG. 8 indicating the RBA assays of hVEGFA(165 and 121), mVEGFA (120) on human VEGFR2: (A) RBA assays of hVEGFA(165) on human VEGFR2 showed IC50 of Aflibercept, SLN6071, SLN6073, SLN6075, Bevacizumab are 53.5 pM, 36.7 pM, 40.9 pM, 25.3 pM, 4494.6 pM, respectively. (B) RBA assays of hVEGFA(121) on human VEGFR2 showed IC50 of Eylea, SLN6071, SLN6073, SLN6075, Bevacizumab are 1784.3 pM, 907 pM, 854.1 pM, 720.7 pM, 25695.1 pM, respectively. (C) RBA assays of mVEGFA(120) on human VEGFR2 showed SLN6073 and Aflibercept can block VEGFA/VEGFR2 binding. IC50 of Aflibercept and SLN6073 are 859.9 pM, 10267 pM, respectively.

### Example 3 Inhibition of HUVEC Proliferation

Materials: HUVEC cells; EMC (Sclencell, 1001); Cell counting kit-8 (Donjndo)
Procedure: 1). Seed 3×10³ HUVEC cell per well in 96-well plates in complete medium (5% FBS, 1% EGFS, 1% P/S), and culture overnight; 2) Wash the well with 100 μL PBS, and starve the cell with the basal medium for 2 hrs; 3) Serially dilute the test articles (VEGF inhibitors) from 20000 nm/mL to 0.00128 nm/mL with basal medium (containing 1.0% FBS), mix and incubate with same volume of 100 ng/mL VEGF for 4 hrs; 4) Add 100 μL of the mixture as above onto the starved HUVEC cells, and continue culturing for 72 hrs; 5) Add 10 μL CCK-8 and incubate for 3 hrs; 6) Scan the plate at 450 nm; 7) calculate cell viability (%)=[(As−Ab)/(Ac−Ab)]×100; Inhibition rate (%)=[(Ac−As)/(Ac−Ab)]×100; As=OD450 of the experimental well, Ab=OD450 of the blank control, Ac=control well absorbance.
The results are shown in FIG. 9 indicating the inhibition of HUVEC cell proliferation. SLN6073 showed about three-fold stronger inhibition ability than Aflibercept. Bi-specific VHH-Fcs SLN6073, SLN6071 are better than parent mono-VHH-Fcs SLN6008, SLN6005, SLN6068 and SLN6043.

### Example 4 Dual Functional Recombinant Proteins Inhibiting VEGF-Driven Angiogenesis and Factor H-Regulated Complement Activation

Truncated CFH (domain 1-4 and 19-20) was fused to the C-terminus of SLN6073 (VHH-Fc) to form SLN6074 (VHH-Fc-CFH) to make a dual functional recombinant protein inhibiting VEGF-driven angiogenesis and factor H-regulated complement activation. The results are shown in FIG. 11. Left panel of (A) and (C) shows binding activity of Bi-specific VHH-Fc-CFH protein (SLN6074), parent VHH-Fc-CFH protein (SLN6073) and SLN6066 (Aflibercept) to hVEGFA121-Biotin and mVEGFA120-Biotin. Proteins were 5-fold serially diluted from 2 μg/ml and the binding to coated hVEGFA121-Biotin was detected by goat anti-human Fc-HRP. Right panel of (B) and (D) shows receptor blocking activity of Bi-specific VHH-Fc-CFH protein (SLN6074), parent VHH-Fc-CFH protein (SLN6073) and SLN6066 (Aflibercept). 50 μl hVEGFA121-biotin (2 μg/ml) and 50 μl 5-fold serially diluted proteins (5 μg/ml) were mixed and its binding to coated 5 μg/ml fusion proteins was detected by SA-HRP. (E) shows the inhibition of hemolysis. SLN6074 (VHH-Fc-CFH) demonstrated favorable binding to human and mouse VEGFA and also comparable activities to that of VHH-Fc regarding blocking the VEGFR-VEGF interaction, as assessed with the purified recombinant proteins by following procedures as described above. Complement inhibition activity was determined by following a standard procedure for measuring alternative pathway functions on red blood cells (hemolysis assay). SLN6074 (VHH-Fc-CFH) has comparable hemolysis inhibition to SLN7112(Fc-CFH). Such dual functional protein provides a potential therapeutical strategy for AMD or tumor.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An isolated antigen-binding protein specifically binding to VEGFA, comprising a HCDR3, a HCDR2 and a HCDR1;
wherein said HCDR3 comprises the amino acid sequence of SEQ ID NO: 1, SEQ ID NO: 4, SEQ ID NO: 7 or SEQ ID NO: 10; said HCDR2 comprises the amino acid sequence of SEQ ID NO: 2, SEQ ID NO: 5, SEQ ID NO: 8 or SEQ ID NO: 11; and said HCDR1 comprises the amino acid sequence of SEQ ID NO: 3, SEQ ID NO: 6, SEQ ID NO: 9 or SEQ ID NO: 12.

2-3. (canceled)

4. The isolated antigen-binding protein of claim 1, wherein said isolated antigen-binding protein comprises antibody or its antigen-binding fragment.

5. The isolated antigen-binding protein of claim 4, wherein said antibody comprises single domain antibody, monoclonal antibody, single strand antibody, chimeric antibody, polyspecific antibody, humanized antibody and fully human antibody.

6. The isolated antigen-binding protein of claim 4, wherein said antigen-binding fragment comprises Fab, Fab′, F(ab)2, F(ab′)2, sdAb, Fv and ScFv fragment and bi-paratopic antigen-binding protein.

7. The isolated antigen-binding protein of claim 4, wherein said isolated antigen-binding protein is a single domain antibody (sdAb) or its antibody fragment.

8-10. (canceled)

11. The isolated antigen binding protein of claim 1, comprising a HCDR1, a HCDR2, and a HCDR3, wherein said HCDR1, said HCDR2 and said HCDR3 comprise the amino acid sequences selected from the group consisting of:
1) said HCDR1 comprises the amino acid sequence of SEQ ID NO: 3, said HCDR2 comprises the amino acid sequence of SEQ ID NO: 2, and said HCDR3 comprises the amino acid sequence of SEQ ID NO: 1;
2) said HCDR1 comprises the amino acid sequence of SEQ ID NO: 6, said HCDR2 comprises the amino acid sequence of SEQ ID NO: 5, and said HCDR3 comprises the amino acid sequence of SEQ ID NO: 4;
3) said HCDR1 comprises the amino acid sequence of SEQ ID NO: 9, said HCDR2 comprises the amino acid sequence of SEQ ID NO: 8, and said HCDR3 comprises the amino acid sequence of SEQ ID NO: 7; and
4) said HCDR1 comprises the amino acid sequence of SEQ ID NO: 12, said HCDR2 comprises the amino acid sequence of SEQ ID NO: 11, and said HCDR3 comprises the amino acid sequence of SEQ ID NO: 10.

12. The isolated antigen-binding protein claim 1, comprising the amino acid sequence of any one of SEQ ID NO: 13-16.

13. A bi-paratopic antigen-binding protein, comprising a first antigen-binding domain, and a second antigen-binding domain, wherein said first antigen-binding domain and/or said second antigen binding domain comprises said antigen-binding protein of claim 1.

14. The bi-paratopic antigen-binding protein of claim 13, wherein said first antigen-binding domain and said second antigen-binding domain target the same antigen.

15. The bi-paratopic antigen-binding protein of claim 13, comprising a HCDR3, said HCDR3 comprises the amino acid sequence of SEQ ID NO: 1, SEQ ID NO: 4, SEQ ID NO: 7 or SEQ ID NO: 10.

16. The bi-paratopic antigen-binding protein of claim 13, comprising the amino acid sequence of any one of SEQ ID NO: 17-19.

17. A polypeptide, comprising the isolated antigen-binding protein of claim 1.

18. The polypeptide of claim 17, further comprising at least one other functional domain.

19. The polypeptide of claim 18, wherein said at least one other functional domain comprises a human IgG Fc region.

20-21. (canceled)

22. The polypeptide of claim 18, wherein said at least one other functional domain comprises a human complement H (CFH) fragment.

23. The polypeptide of claim 17, comprising the amino acid sequence of SEQ ID NO: 21 or SEQ ID NO: 23.

24. An isolated nucleic acid molecule, encoding the isolated antigen binding protein of claim 1.

25. A vector, comprising the nucleic acid molecules of claim 24.

26. (canceled)

27. A pharmaceutical composition, comprising the isolated binding-protein of claim 1.

28-29. (canceled)

30. A method of preventing or treating a disease comprising administering the isolated binding-protein of claim 1 to a subject in need thereof.


SEQUENCE LISTING (38 sequences)

SEQ ID NO: 1 - protein, 18 aa, Artificial Sequence
  SLN-6068(C2-11) CDR3
RMSRLLGMAPLLPEHYDS

SEQ ID NO: 2 - protein, 17 aa, Artificial Sequence
  SLN-6068(C2-11) CDR2
AAYAGGGGTVYADSVKG

SEQ ID NO: 3 - protein, 10 aa, Artificial Sequence
  SLN-6068(C2-11) CDR1
VYTSSTYYMA

SEQ ID NO: 4 - protein, 18 aa, Artificial Sequence
  SLN-6043 (F6-1) CDR3
KWPCTYRRGGSAYRPAVV

SEQ ID NO: 5 - protein, 16 aa, Artificial Sequence
  SLN-6043 (F6-1) CDR2
SISSDDSTRYADSVKG

SEQ ID NO: 6 - protein, 10 aa, Artificial Sequence
  SLN-6043 (F6-1) CDR1
GFTSDNCAVH

SEQ ID NO: 7 - protein, 18 aa, Artificial Sequence
  SLN-6062 (C8-9) CDR3
TWSWYSFTCPGDQNAYIH

SEQ ID NO: 8 - protein, 17 aa, Artificial Sequence
  SLN-6062 (C8-9) CDR2
TLNRNSGRTSYVGSVKG

SEQ ID NO: 9 - protein, 8 aa, Artificial Sequence
  SLN-6062 (C8-9) CDR1
VYTASCMG

SEQ ID NO: 10 - protein, 15 aa, Artificial Sequence
  SLN-6065 (D2-9) CDR3
GWRGGSFWTPSKYSY

SEQ ID NO: 11 - protein, 17 aa, Artificial Sequence
  SLN-6065 (D2-9) CDR2
SIATNTGNDYYADSVKG

SEQ ID NO: 12 - protein, 10 aa, Artificial Sequence
  SLN-6065 (D2-9) CDR1
RDAYFNNYMA

SEQ ID NO: 13 - protein, 127 aa, Artificial Sequence
  SLN-6068(C2-11)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSS

SEQ ID NO: 14 - protein, 126 aa, Artificial Sequence
  SLN-6043 (F6-1)
EVQLVESGGGVVQPGGSLRLSCAASGFTSDNCAVHWVRQAPGKGLEWVSSISSDDSTRYADSVKGRFTISRDNSKNSLYLQMNSLRTEDTALYYCQTKWPCTYRRGGSAYRPAVVWGQGTLVTVSS

SEQ ID NO: 15 - protein, 125 aa, Artificial Sequence
  SLN-6062 (C8-9)
EVQLVESGGGLVQPGGSLRLSCAASVYTASCMGWFRQAPGKGLELVSTLNRNSGRTSYVGSVKGRFTISQDNSKKTLYLQMNSLRAEDTAVYYCAATWSWYSFTCPGDQNAYIHWGQGTLVTVSS

SEQ ID NO: 16 - protein, 124 aa, Artificial Sequence
  SLN-6065 (D2-9)
EVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSS

SEQ ID NO: 17 - protein, 268 aa, Artificial Sequence
  (SLN6071, bi-paratopic VHH-1)C2_F6
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGVVQPGGSLRLSCAASGFTSDNCAVHWVRQAPGKGLEWVSSISSDDSTRYADSVKGRFTISRDNSKNSLYLQMNSLRTEDTALYYCQTKWPCTYRRGGSAYRPAVVWGQGTLVTVSS

SEQ ID NO: 18 - protein, 267 aa, Artificial Sequence
  (SLN6075, , bi-paratopic VHH-2)C2_C8
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASVYTASCMGWFRQAPGKGLELVSTLNRNSGRTSYVGSVKGRFTISQDNSKKTLYLQMNSLRAEDTAVYYCAATWSWYSFTCPGDQNAYIHWGQGTLVTVSS

SEQ ID NO: 19 - protein, 266 aa, Artificial Sequence
  (SLN6079, bi-paratopic VHH-3)C2_D2
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSS

SEQ ID NO: 20 - protein, 229 aa, Artificial Sequence
  Fc region
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 495 aa, Artificial Sequence
  (SLN6073, C2_D2-Fc fusion)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 373 aa, Artificial Sequence
  CFH
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEESTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 23 - protein, 873 aa, Artificial Sequence
  (SLN6074, C2_D2-Fc-CFH fusion)
EVQLVESGGGLVQPGGSLRLSCAASVYTSSTYYMAWFRQAPGKGREGVAAAYAGGGGTVYADSVKGRFTISQDNSKNTLYLQMNSLRAEDTAVYYCAARMSRLLGMAPLLPEHYDSWGQGTLVTVSSGGGGSGGGGSGGGGSEVQLVESGGGLVQPGGSLRLSCAASRDAYFNNYMAWFRQAPGKGLEGVSSIATNTGNDYYADSVKGRFTISRDNSKNTIYLQMNSLRAEDTAVYYCAAGWRGGSFWTPSKYSYWGQGTLVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEESTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 24 - protein, 25 aa, Artificial Sequence [sequence omitted]

SEQ ID NO: 25 - protein, 14 aa, Artificial Sequence
  SLN-6068(C2-11) FR2
WFRQAPGKGREGVA

SEQ ID NO: 26 - protein, 32 aa, Artificial Sequence
  SLN-6068(C2-11) FR3
RFTISQDNSKNTLYLQMNSLRAEDTAVYYCAA

SEQ ID NO: 27 - protein, 11 aa, Artificial Sequence
  SLN-6068(C2-11) /SLN-6062(C8-9) /SLN-6065(D2-9) /SLN6043(F6-1) FR4
WGQGTLVTVSS

SEQ ID NO: 28 - protein, 25 aa, Artificial Sequence
  SLN-6043 (F6-1) FR1
EVQLVESGGGVVQPGGSLRLSCAAS

SEQ ID NO: 29 - protein, 14 aa, Artificial Sequence
  SLN-6043 (F6-1) FR2
WVRQAPGKGLEWVS

SEQ ID NO: 30 - protein, 32 aa, Artificial Sequence
  SLN-6043 (F6-1) FR3
RFTISRDNSKNSLYLQMNSLRTEDTALYYCQT

SEQ ID NO: 31 - protein, 14 aa, Artificial Sequence
  SLN-6062 (C8-9) FR2
WFRQAPGKGLELVS

SEQ ID NO: 32 - protein, 32 aa, Artificial Sequence
  SLN-6062 (C8-9) FR3
RFTISQDNSKKTLYLQMNSLRAEDTAVYYCAA

SEQ ID NO: 33 - protein, 14 aa, Artificial Sequence
  SLN-6065 (D2-9) FR2
WFRQAPGKGLEGVS

SEQ ID NO: 34 - protein, 32 aa, Artificial Sequence
  SLN-6065 (D2-9) FR3
RFTISRDNSKNTIYLQMNSLRAEDTAVYYCAA

SEQ ID NO: 35 - protein, 25 aa, Artificial Sequence
  SLN-6068(C2-11) FR1; SLN-6062 (C8-9) FR1; SLN-6065 (D2-9) FR1; SLN-6043 (F6-1) FR1
  misc_feature (11)..(11): Xaa = Leu or Val
EVQLVESGGGXVQPGGSLRLSCAAS

SEQ ID NO: 36 - protein, 14 aa, Artificial Sequence
  SLN-6068(C2-11) FR2; SLN-6043 (F6-1) FR2; SLN-6062 (C8-9) FR2; SLN-6065 (D2-9) FR2
  misc_feature (2)..(2): Xaa = Phe or Val
  misc_feature (10)..(10): Xaa = Leu or Arg
  misc_feature (12)..(12): Xaa = Gly, Leu or Trp
  misc_feature (14)..(14): Xaa = Ala or Ser
WXRQAPGKGXEXVX

SEQ ID NO: 37 - protein, 32 aa, Artificial Sequence
  SLN-6068(C2-11) FR3; SLN-6043 (F6-1) FR3; SLN-6062 (C8-9) FR3; SLN-6065 (D2-9) FR3
  misc_feature (6)..(6): Xaa = Gln or Arg
  misc_feature (11)..(11): Xaa = Lys or Asn
  misc_feature (12)..(12): Xaa = Ser or Thr
  misc_feature (13)..(13): Xaa = Ile or Leu
  misc_feature (22)..(22): Xaa = Ala or Thr
  misc_feature (27)..(27): Xaa = Leu or Val
  misc_feature (31)..(31): Xaa = Ala or Gln
  misc_feature (32)..(32): Xaa = Ala or Thr
RFTISXDNSKXXXYLQMNSLRXEDTAXYYCXX

SEQ ID NO: 38 - protein, 429 aa, Artificial Sequence
  SLN-6066
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK